(19) United States
(12) Patent Application Publication
(10) Pub. No.: US 2008/0255407 A1
(43) Pub. Date: Oct. 16, 2008

(54) Bi-directional system for dissecting and harvesting vessels

(75) Inventors: Derek C. Blakeney, Saline, MI (US); Randal James Kadykowski, South Lyon, MI (US); David B. Maurer, Ann Arbor, MI (US)

Correspondence Address: TERUMO CARDIOVASCULAR SYSTEMS CORPORATION;ATTN: GAEL DIANE TISACK, 6200 JACKSON ROAD, ANN ARBOR, MI, 48103, US

(73) Assignee: Terumo Cardiovascular Systems Corporation

(21) Appl. No.: 11/786,113

(22) Filed: Apr. 11, 2007

(51) Int. Cl. A61F 2/06

(52) U.S. Cl. 600/36

(57) ABSTRACT

A method and apparatus for bi-directionally dissecting and harvesting a vessel includes a dissector device that is at least temporarily secured to a harvester device.

### CROSS-REFERENCE TO RELATED APPLICATIONS AND STATEMENT REGARDING SPONSORED RESEARCH

Not applicable.

### BACKGROUND OF THE INVENTION

The present invention relates to the harvesting of blood vessels and, more particularly, to a method and apparatus for dissection and removal of sections of blood vessels.

The harvested vessels are used in many surgical procedures, including use as a coronary artery bypass graft, or in other cardiovascular procedures. As one example, in vascular and cardiovascular procedures, a blood vessel or vessel section, such as an artery or vein, is “harvested” (i.e., removed) from its natural location in a patient's body and is used elsewhere in the body. For example, in coronary artery bypass, grafting surgery, the harvested blood vessel is used to form a bypass between an arterial blood source and one or more coronary arteries. Among the preferred sources for the vessels to be used as the bypass graft are the saphenous vein in the leg and the radial artery in the arm.

Endoscopic surgical procedures for harvesting a section of a blood vessel (e.g., the saphenous vein) subcutaneously have been developed in order to avoid disadvantages and potential complications of harvesting of the blood vessel. In the past, the harvesting was done through a continuous incision (e.g., along the leg) that exposed the full length of the desired vein section. The continuous incision had been necessary in order to provide adequate exposure for visualizing the vein and for introducing the surgical instruments to seal and sever the tissue and side branches of the vessel.

A more recent development has been a minimally-invasive technique that employs a small incision for locating the desired vessel and for introducing one or more endoscopic devices into the small incision. For example, commercially available products for performing the endoscopic blood vessel harvesting procedure include a number of separate endoscopic devices that are each inserted into the patient. These endoscopic products include, for example, an insufflation mechanism having plastic tubing to supply air or CO₂ to insufflate the subcutaneous area; an endoscope having a camera and light cables in order to visualize both the dissection and harvesting procedures; a dissector mechanism to dissect or separate the vessel from surrounding tissues in the body; and a harvester mechanism to seal and sever any branches from the vessel and to remove the vessel from the body. In certain instances, the combination of mechanisms can be bulky and cumbersome for the clinician performing the vessel harvesting. Also, in certain instances, these mechanisms require that a relatively large diameter wound and cavity be formed within the patient in order to accommodate all the separate mechanisms.

Since there is an increasing occurrence of minimally invasive surgery, there is also a growing need for more efficient and compact devices that shorten the time and lessen the invasiveness of the surgery.

It would be especially useful to have a method for dissecting and harvesting vessel that provides a still less invasive surgery. It would also be desirable to have a device that allows for efficient maneuvering within the body and that causes fewer intrusions into the open wound in the patient.

It would also be desirable to have a dissector/harvester device that is compact and does not require multiple insertions into the patient.

### SUMMARY OF THE INVENTION

In one aspect, there is provided a method for bi-directionally dissecting and harvesting a vessel. The method includes:

inserting at least a distal end of a dissector device into a first wound in the body, the dissector device including a dissector connecting member;

advancing at least the distal end of the dissector device alongside the vessel to be dissected and harvested from the body in a direction toward a second wound in the body to form a cavity substantially surrounding the vessel;

positioning a harvester device adjacent to the dissector distal end, the harvester device having a distal end that includes a harvester connecting member;

at least temporarily securing the harvester distal end to the dissector distal end to form a temporarily connected dissector-harvester device;

advancing the at least temporarily connected harvester-dissector device in the cavity alongside the vessel in a direction back toward the first wound; and

at least intermittently activating the harvester device to seal and sever any branches extending from the vessel.

The harvester device is at least intermittently activated to seal and sever any branches extending from the vessel. In certain embodiments, the dissector device and the harvester device are in an opposed axial alignment when the harvester device is being advanced toward the first wound.

In another aspect, there is provided a bi-directional dissecting and harvesting device. The dissector device has a dissector distal end that is configured to be at least partially inserted in a first wound in the body. The harvester device is configured to be at least partially inserted into a second wound in the body. The harvester device has a harvester distal end that is configured to be at least temporarily secured to the dissector distal end. Additionally, the harvester device includes a sealing and severing device for sealing and severing branches extending from the vessel.

Various objects and advantages of this invention will become apparent to those skilled in the art from the following detailed description of the preferred embodiment, when read in light of the accompanying drawings.

### BRIEF DESCRIPTION OF THE DRAWINGS

FIG. 1 is a structure diagram showing a dissector positioned adjacent to a first wound and a harvester device positioned adjacent to a second wound.

FIG. 2 is a structure diagram, partially in phantom, showing the dissector device positioned adjacent to the second wound.

FIG. 3 is a structure diagram, partially in phantom, showing the harvester device positioned adjacent to the first wound.

FIG. 4 is a structure diagram of one embodiment, partially in phantom, showing a distal end of a dissector device and a distal end of a harvester device.

FIG. 5 is a structure diagram of another embodiment, partially in phantom, showing a distal end of a dissector device and a distal end of a harvester device.

### DETAILED DESCRIPTION OF THE PREFERRED EMBODIMENTS

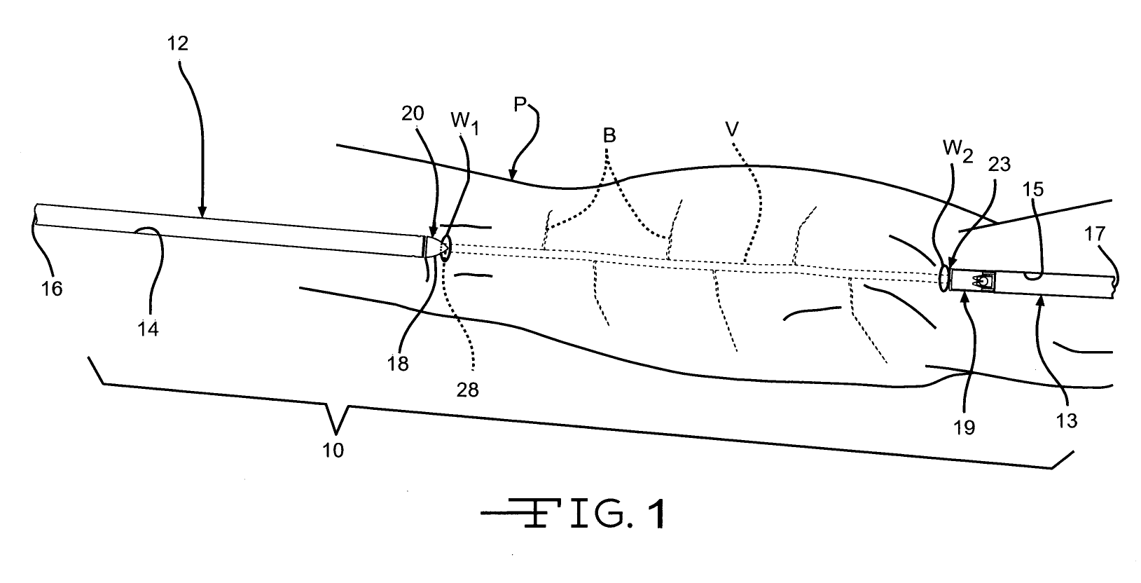

In one aspect, there is provided herein a method for bi-directionally dissecting and/or harvesting a vessel from a patient's body. Referring first to FIGS. 1, 2 and 3, structure diagrams are provided that schematically illustrates a dissecting phase and a harvesting phase of the inventive bi-directional dissecting-harvesting method. It is to be understood that, in one aspect of the present invention, the illustrations and the devices described herein are shown for purposes of illustration and it is within the contemplated scope of the present invention described herein that other suitably configured devices can be used to perform the bi-directional dissecting-harvesting method. As such, in the FIGS. 1, 2 and 3, a bi-directional dissector-harvester device 10 is generally schematically illustrated for showing how a method for bi-directionally dissecting and harvesting of a vessel from a body can be accomplished. The bi-directional dissector-harvester device 10 is shown as having a dissector device 12 and a harvester device 13.

FIG. 1 shows a structure diagram showing one non-limiting embodiment of a suitable bi-directional dissector/harvester device 10 for bi-directionally dissecting and/or harvesting a vessel V and for sealing and severing branches B from the vessel V. The dissector/harvester device 10 includes the elongated dissector device 12 for at least partial insertion in the body P through the first wound W₁, or cut skin portion, and the elongated harvester device 13 for at least partial insertion into a body through a second wound W₂, or cut skin portion.

The dissector device 12 includes a dissector sheath 14 that has a proximal end 16 and a dissector distal end 18. In the embodiment shown, the dissector distal end 18 includes a dissector connecting member 20, as will be further explained below. In the embodiment illustrated, the dissector connecting member 20 is axially positioned at the dissector distal end 18. In certain embodiments, at least a portion of the dissector connecting member 20 is transparent in order to allow visualization of the vessel V and the surrounding tissue.

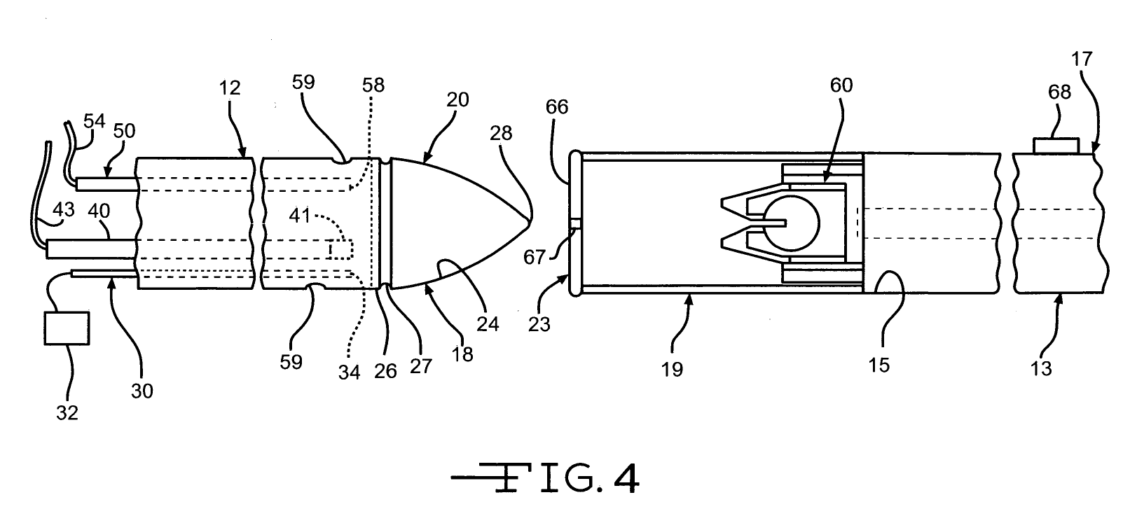

As best seen in FIG. 4, the dissector connecting member 20 defines an inner space 24 and includes a base portion 26 and a dissecting tip 28. The dissecting tip 28 is spaced apart from the base portion 26. In the non-limiting embodiment shown in the FIGURES herein, the dissecting tip 28 of the dissector connecting member 20 can have a conical or other tapered shape to aid in the harvesting, of a vessel.

The harvester device 13 includes a harvester sheath 15 that has a proximal end 17 and harvester distal end 19. In the embodiment shown, the harvester device 13 includes a harvester connecting member 23.

In the non-limiting embodiment shown in the FIGURES, the harvester connecting member 23 is operatively connected to the connecting member 20 of the dissector device 12. In one non-limiting embodiment, as illustrated in FIG. 4, the base portion 26 of the dissector connecting member 20 can have a stepped edge 27 that allows the base portion 26 to be at least partially co-axially aligned within the harvester device 13.

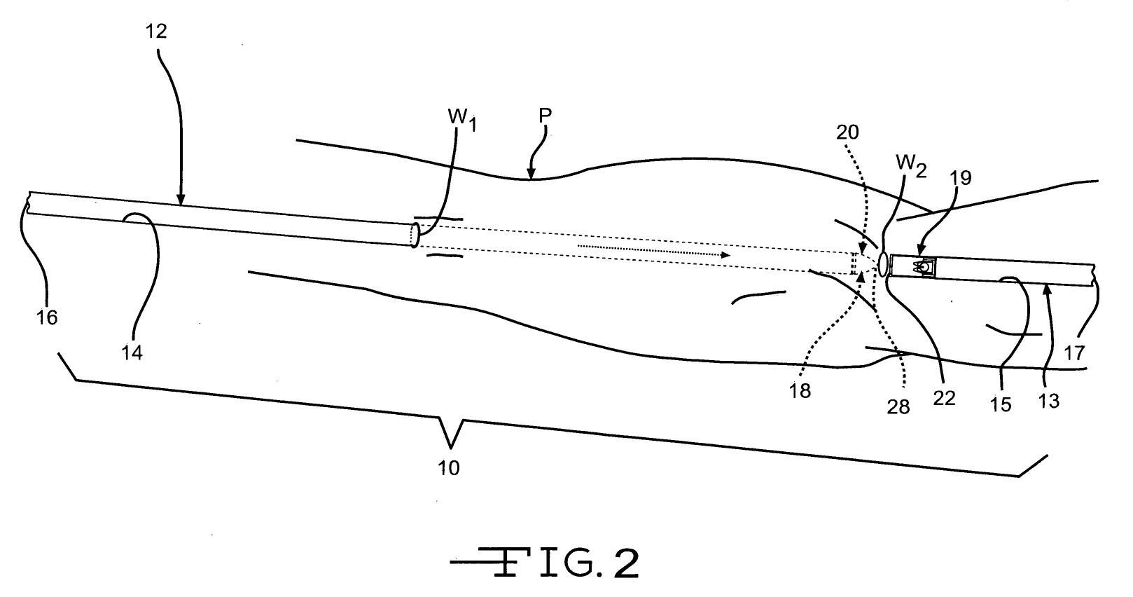

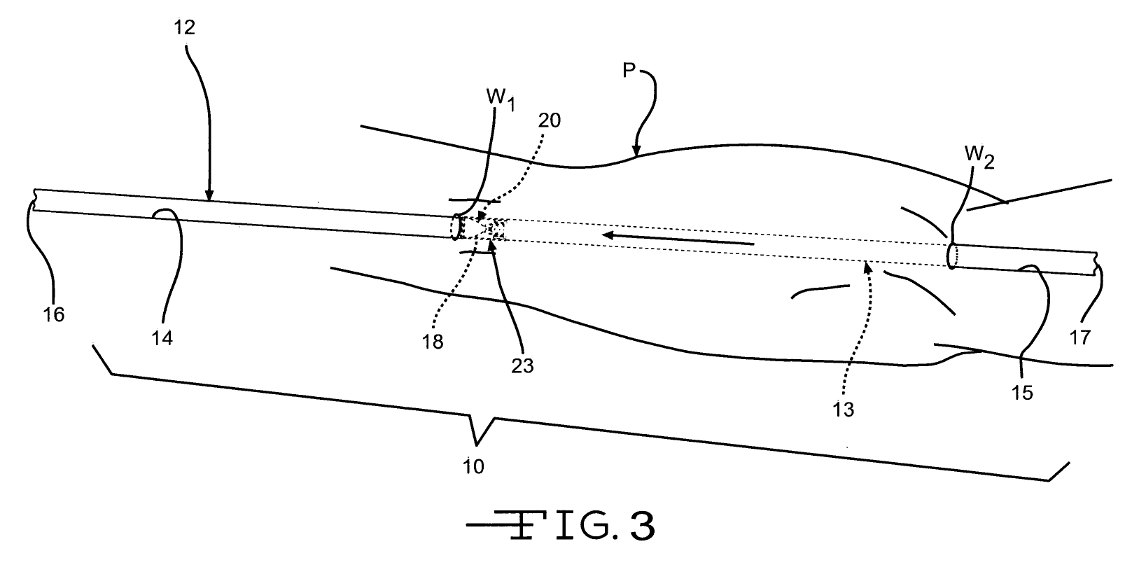

During a harvesting phase of the method, as shown in FIG. 3, the dissector connecting member 23, which now forms a part of the at least temporarily connected bi-directional harvester-dissector device 10, is at least partially inserted into a first wound W₁ in the body P. The distal end 18 of the dissector device 12 is advanced alongside the vessel V in a direction toward a second wound W₂ in the body P, as schematically illustrated in FIG. 2. The dissector distal end 18 forms a cavity substantially surrounding the vessel V as the dissector device is advanced by a clinician toward a second wound W₂.

Once the dissector device 12 has been advanced by the clinician to a point adjacent to the second wound W₂, the clinician then aligns the harvester device 13 with the dissector device 12.

In order to initiate the harvesting phase of the bi-directional dissecting/harvesting method, the harvester device 13 is positioned at a point near the second wound W₂. The harvester device 13 is advanced by the clinician toward the dissector device 12 until the harvester device 13 and the dissector device 12 are brought into at least temporary engagement or connection.

The harvester device 13 is at least temporarily connected to the dissector device 12; thus at least temporarily forming the bi-directional dissector-harvester device 10. It is to be understood, that in certain embodiments, the dissector distal end 18 can be extended out of the second wound W₂ to allow the clinician to readily align the dissector distal end 18 with the harvester distal end 19.

During the harvesting phase of the method, as shown in FIG. 3, the at least temporarily connected bidirectional harvester-dissector device 10 is retracted in the cavity alongside the vessel in a direction back toward the first wound W₁. The harvester device 13 is at least intermittently activated to seal and sever any branches B extending from the vessel V.

The harvester connecting member 23 on the harvesting distal end 19 can have any desired shape that allows the dissector device 12 and the harvester device 13 to be connected. In certain embodiments, the harvester device 13 can have a cross-sectional diameter that is substantially the same as the cross-sectional diameter of the dissector device 12. In other embodiments, the harvester device 13 can have a diameter that is smaller than the dissector device 12.

It is also to be understood that the distal ends 18 and 19 of the dissector device 12 and the harvester device 13, respectively, can be connected in any suitable manner. The distal ends 18 and 19 can be suitably configured to allow for easy connection and easy disconnection. In certain non-limiting examples of suitable connection mechanisms, the distal ends 18 and 19 can include one or more of: interlocking surfaces, interlocking members, threaded engagement members, and the like. In another non-limiting example, there can be a magnetic connection between the distal ends 18 and 19 where the magnetic attraction can be continuous or intermittent. It is to be understood that other suitable mechanisms for at least temporarily connecting the dissector device 12 and the harvester device 13 are within the contemplated scope of the present invention.

In one embodiment, as shown in FIG. 4, the harvester connecting member 23 includes a connection member 66. The connection member 66 is at least partially co-axially positioned around the dissector connector member 20 to at least temporarily connect the harvester device 13 and the dissector device 12. Also, in certain embodiments, the connection member 66 defines an open space or gap 67 that allows the connection member 66 to expand during the positioning of the connection member 66 over the dissecting tip 28. During the practice of the bi-directional dissecting/harvesting method, the connection member 66 is brought into mating engagement with the detent 27 on the dissector connector member 20.

Also, the harvester connecting member 23 can be linked to a displacing button 68 that advances and returns the connection member 66 in an axially extending longitudinal direction toward and/or away from the dissector device 12.

In one non-limiting embodiment, the harvesting device 13 can include a sealing and severing device 60 for sealing and severing the branches B from the vessel V. The sealing and severing tool 60 includes a cauterizing tool, such as, for example, a bipolar electrocautery tool or an ultrasonic cauterizing tool.

Thus, the dissector device 12 is gradually withdrawn from the first wound W₁ as the harvester device 13 is advanced toward the first wound W₁. Once at least the dissector distal end 18 is either adjacent to, or removed from, the first wound W₁, the harvester distal end 19 can be disconnected from the dissector distal end 18. The harvester device 13 is then removed from the body P by being retracted in a direction back toward the second wound W₂.

In certain embodiments, the bi-directional dissector/harvester device 10 can include a light source 30 that is axially positioned within the dissector sheath 14 of the dissector device 12. The light source 30 includes a power supply 32 and a light 34. In certain embodiments, the power supply 32 is a suitable battery-type power source that can be remotely controlled to be in an “on” or illuminating mode, or in an “off” or non-illuminating mode. In certain embodiments, the light source 30 is slidably positioned within the dissector sheath 14 of the dissector device 12 and can be removed from the dissector device 12. In other embodiments, the light source 30 is incorporated into the imaging system 40.

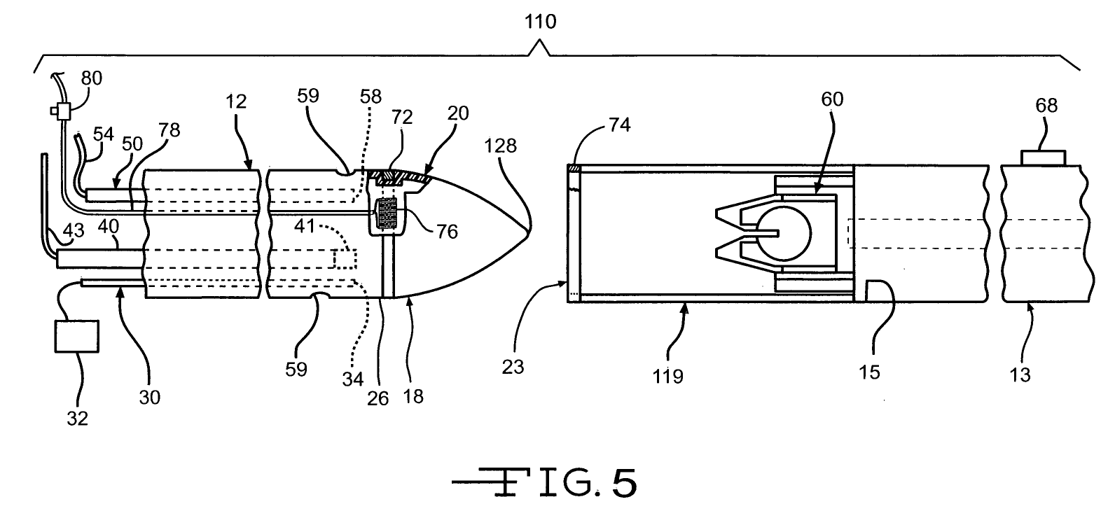

The bi-directional dissector/harvester device 10 can also include an imaging system 40 that is axially positioned within the dissector sheath 14 of the dissector 12. In the non-limiting embodiment shown in the FIGS. 4 and 5 herein, the imaging system 40 is positioned at least adjacent to the dissecting tip 28. The imaging system 40 includes a suitable image-receiving device 41 that converts images into signals for transmission, recording and/or storage, and/or takes photographs of such images, such as a camera. During the initial use of the bi-directional dissector/harvester device 10, the imaging system 40 is oriented so that an image through the dissecting tip 28 is visible when the vessel V is being-harvested and separated from the surrounding tissue. In certain embodiments, the imaging device 41 can be fixedly oriented in a forward direction, as defined by facing from the first wound WI toward the second wound W₂.

It should be understood, that various suitable mechanisms can be used for directing the movement of the camera 41 and/or for receiving the images from the imaging system 40. For example, in the embodiment shown in FIG. 1, the camera 41 is operatively connected to a suitable cable 43. The cable 43 can be connected to a suitable viewing monitor (not shown). In other embodiments, the camera 41 can be a wireless device that transmits images.

In certain embodiments, the bidirectional dissector/harvester device 10 can further include an insufflation device 50. In one such embodiment, the insufflation device 50 is axially positioned within the dissector sheath 14 of the device 12. The insufflation device 50 provides a supply of a suitable gas through a supply line 54 having a discharge end 58. In certain embodiments, the discharge end 58 of the supply line 54 is located near the distal end 18 of the dissector device 12. The dissector device 12 can include one or more discharge ports or holes 59 that are in communication with the dissector sheath 14. The gas escaping from the ports 59 forms the cavity/tunnel in the body and keeps the surrounding tissue away from the vessel V. The insufflation device 50 allows gas to be delivered via the radially extending openings 59 in the dissector device 14. The gas is delivered in a suitable manner subcutaneously to an area adjacent to the vessel V to be dissected and harvested.

In the practice of the method described herein, two wounds, W₁ and W₂, are formed in the patient, as generally shown in FIGS. 1, 2 and 3. To begin the dissection procedure, the dissecting tip 28 of the dissector device 12 is inserted through the first wound WI in the patient. In operation, the dissecting tip 28 is pressed into the tissues surrounding the vessel V, thereby forming a tunnel or cavity around the vessel V. In certain embodiments, it is desired that the dissecting tip 28 be pressed into the surrounding tissue generally along the direction of the vessel V in order to dissect, or separate, the vessel V from adjacent tissue without damage to the vessel or surrounding tissue.

Upon inserting the dissector device 12 under the patient's skin, it is possible to obtain an image illuminated by the illuminating light 34 from the light source 30. Also, in certain embodiments, the insufflation device 50 is activated for inflating the area adjacent the vessel as the cavity is being formed.

The dissecting tip 28 is used to perform an initial, or blunt, dissection of the vessel from the surrounding tissue. The clinician continues to perform the dissection of the vessel V from the surrounding tissue, advancing the dissector device 12 toward the second wound W₂. Once the dissecting tip 28 is at least adjacent to the second wound W₂, the harvester device 13 is brought into alignment with the dissector device 12. In the non-limiting embodiment shown, the connection member 66 is advanced by the clinician over the dissecting tip 28 to at least a point where the connection member 66 is engaged by or seated within the detent 27 on the dissecting connecting member 20. The detent 27 is configured to receive at least a portion of the connection member 66. In one non-limiting embodiment, as shown in FIG. 4, the detent 27 is configured to allow the connection member 66 to be quickly and readily snapped into the detent 27 so that there can be a quick and easy alignment of the dissector device 12 and the harvester device 13.

The bi-directional harvesting device 10 is now configured in a unitary manner such that both the dissector device 12 and the harvester device 13 are simultaneously withdrawn or moved back in a direction toward the first wound W₁.

The clinician can view the harvesting through the imaging system 40. In other embodiments, the harvester device 13 can include an endoscope, if desired.

As the clinician moves the now connected bi-directional dissector/harvester device 10 back along the formed cavity alongside the vessel V, the harvesting device 60 is at least intermittently activated to seal and sever any branches B that extend from the vessel V. The imaging device 40 and/or the insufflation device 50 can also be activated so that the clinician has a clear view of the branches being sealed and severed. It is to be noted that the method described herein reduces the invasiveness of the harvesting procedure since there is no need to re-inflate the formed cavity. The dissector device 12 maintains the formed cavity in an expanded condition as the-harvester device 13 is being engaged.

FIG. 5 shows another embodiment of a bidirectional dissector-harvester device 110. In the embodiment shown, the dissector device 12 and the harvester device 13 are illustrated as having the same general elements as for the embodiment shown in FIG. 4. It is to be understood, however, that in certain embodiments, it may be desired that the dissector device 12 and the harvester device 13 can each include other elements and/or have different configurations. For ease of illustration herein, however, the common elements in the dissector device 12 and the harvester device 13 are shown herein as having the numeral, and only the different elements will be described in detail.

The dissector device 12 has a dissector connection member 20 having a base portion 26 and dissecting tip 128. The base portion 26 of the dissector connecting member 20 includes a first magnetic member 72 that allows the base portion 26 to be at least partially co-axially aligned within and secured to the harvester device 13.

The harvester connecting member 23 on the harvesting distal end 119 can have any desired shape that allows the dissector device 12 and the harvester device 13 to be connected. In the embodiment shown in FIG. 5, the harvester connecting member 23 includes a second magnetic member 74 at its distal end. During use, the second magnetic member 74 is at least partially co-axially positioned around the first magnetic member 72 to at least temporarily connect the harvester device 13 and the dissector device 12. Also, in certain embodiments, at least the first magnetic member 72 can be operatively connected via a wire 78 to a power supply 80. The first and second magnetic members 72 and 74 are in a contacting (or, in certain embodiments, locking) engagement and the power supply 80 is activated to cause a magnetic attraction between the first and second magnetic members 72 and 74.

While the invention has been described with reference to various and preferred embodiments, it should be understood by those skilled in the art that various changes may be made and equivalents may be substituted for elements thereof without departing from the essential scope of the invention. In addition, many modifications may be made to adapt a particular situation or material to the teachings of the invention without departing from the essential scope thereof. Therefore, it is intended that the invention not be limited to the particular embodiment disclosed herein contemplated for carrying out this invention, but that the invention will include all embodiments falling within the scope of the claims.

What is claimed is:

1. A method of dissecting and harvesting a vessel from a body, the method comprising:
inserting at least a distal end of a dissector device into a first wound in the body, the dissector device including a dissector connecting member;
advancing at least the distal end of the dissector device alongside the vessel to be dissected and harvested from the body in a direction toward a second wound in the body to form a cavity substantially surrounding the vessel;
positioning a harvester device adjacent to the dissector distal end, the harvester device having a distal end that includes a harvester connecting member;
at least temporarily securing at least the harvester distal end to the dissector distal end to form a temporarily connected dissector-harvester device;
advancing the at least temporarily connected harvester-dissector device in the cavity alongside the vessel in a direction back toward the first wound; and
at least intermittently activating the harvester device to seal and sever any branches extending from the vessel.

2. The method of claim 1, including bringing the dissector distal end and the harvester distal end into at least a temporarily interlocking position.

3. The method of claim 1, including aligning the dissector device and the harvester device in an opposed axial relationship as the harvester device is being connected to the dissector device.

4. The method of claim 1, including extending the dissector distal end out of the second wound and aligning the dissector distal end with the harvester distal end.

5. The method of claim 1, wherein the distal ends of the dissector device and the harvester device are configured to allow for easy connection and easy disconnection.

6. The method of claim 1, wherein the distal ends include one or more of mating surfaces, interlocking members, threaded engagement, and magnetic connections.

7. The method of claim 1, including cauterizing the branches to be severed.

8. The method of claim 1, providing an image of the vessel being dissected and harvested.

9. The method of claim 1, including supplying a gas into the cavity during the advancing of the dissector device and/or during the advancing of the at least temporarily connected dissector-harvester device.

10. The method of claim 1, including using a harvester device that has a smaller cross-sectional diameter than the cross-sectional diameter of the dissector device.

11. A bi-directional dissector-harvester device for dissecting and harvesting a vessel from a body, the bi-directional dissector-harvester device comprising:
a dissector device having a dissector distal end configured to be at least partially inserted in a first wound in a body; and
a harvester device having a harvester distal end configured to be at least temporarily connected to the dissector distal end, the harvester device including a sealing/severing device configured to seal and sever any branches extending from the vessel.

12. The bi-directional dissector-harvester device of claim 11, wherein the dissector distal end and the harvester distal end are configured to be in an opposed axial alignment when the harvester device is connected to the dissector device.

13. The bi-directional dissector-harvester device of claim 1 1, wherein the dissector distal end comprises at least one mating surface configured to at least temporarily mate with at least one mating surface on the harvester distal end.

14. The bi-directional dissector-harvester device of claim 11, wherein the distal ends of the dissector device and the harvester device are configured to allow for easy connection and easy disconnection.

15. The bi-directional dissector-harvester device of claim 11, wherein the one or more of the dissecting distal end and harvesting distal end includes one or more of interlocking surfaces, interlocking members, threaded engagement members, and magnetic connections.

16. The bi-directional dissector-harvester device of claim 11, wherein the severing device is configured to cauterize the branches to be severed.

17. The bi-directional dissector-harvester device of claim 1 1, wherein one or more of the dissector device and harvester device include an imaging system configured to receive images in an area adjacent to the imaging system.

18. The bi-directional dissector-harvester device of claim 11, wherein the dissector device includes an insufflation device configured to supply a gas subcutaneously to an area adjacent to the vessel.

19. The bi-directional dissector-harvester device of claim 11, wherein the harvester device has a smaller cross-sectional diameter than the cross-sectional diameter of the dissector device.
